(19) United States
(12) Patent Application Publication
(10) Pub. No.: US 2024/0184322 A1
(43) Pub. Date: Jun. 6, 2024

(54) A CLOCK OSCILLATOR FOR BIOCHEMICAL REACTION COMPUTERS

(71) Applicant: SOUTH CHINA UNIVERSITY OF TECHNOLOGY, Guangdong (CN)

(72) Inventors: Han Huang, Guangzhou (CN); Chengzhi Ma; Xinglong Xiao, Guangzhou (CN); Tongli He, Guangzhou (CN)

(21) Appl. No.: 18/278,911

(22) PCT Filed: Sep. 24, 2021
(86) PCT No.: PCT/CN2021/120313

§ 371 Date: Aug. 25, 2023

(30) Foreign Application Priority Data
Aug. 9, 2021 (CN) 202110907765.3

(51) Int. Cl. G06F 1/08

(52) U.S. Cl. CPC G06F 1/08

(57) ABSTRACT

The present invention discloses an enzymatic reaction-based clock oscillator for biochemical reaction computers, which uses the concentration or activity of substances or enzymes as a signal, and realizes the self-feedback regulation of the chemical signal through the mutual catalysis of the multiple substances and enzymes, so as to output the periodically changing chemical signal. Compared with the traditional electronic oscillator, the output signal of the clock oscillator provided by the present invention is a chemical signal, so the clock oscillator can be directly used in the biochemical reaction computers without the conversion between an electrical signal and the chemical signal. Compared with the existing DNA-based clock oscillator, because the enzymatic reaction has a faster reaction speed than the DNA replacement reaction and the DNA transcription and translation process, the clock oscillator provided by the present invention can have a higher frequency, thus meeting the requirements of higher-performance biochemical reaction-based numerical operation and logical calculation.

### FIELD OF THE INVENTION

The present invention relates to the field of biochemical computer technology, in particular to an enzymatic reaction-based clock oscillator for biochemical reaction computers.

### BACKGROUND OF THE INVENTION

At present, it is a very potential subject to build a biochemical computer with high performance and low power consumption by following the regulation principle of organisms in nature. In the traditional electronic computers and biochemical computers, the clock oscillator, on which the synchronous timing logic depends, is an important component. The existing clock oscillators based on the DNA cascade displacement reaction and the DNA transcription and translation system have a long clock cycle, usually several hours, because they rely on the slow reactions involving DNA and RNA. This is difficult to meet the needs of building a high-performance biochemical computer, so it is necessary to find a more efficient biochemical reaction and build a new biochemical reaction clock oscillator.

### CONTENTS OF THE INVENTION

The object of the present invention is to solve the defects of slow reaction speed and long clock cycle of the DNA-based biochemical reaction clock oscillator in the prior art by providing an enzymatic reaction-based clock oscillator for biochemical reaction computers, which uses the concentration or activity of substances or enzymes as a clock signal, and drives the concentration and activity of other substances and enzymes to change through the change of enzyme activity, thus forming a negative-feedback enzymatic regulation cycle, so as to realize the spontaneous periodic change of the clock signal.

The object of the present invention can be achieved through the following technical solution:

An enzymatic reaction-based clock oscillator for biochemical reaction computers is provided, comprising a clock signaling enzyme, an anti-clock signaling enzyme, and an output signal.

On the one hand, the concentration or activity of the clock signaling enzyme is affected by that of the anti-clock signaling enzyme: when the concentration or activity of the anti-clock signaling enzyme is high, the generation of the clock signaling enzyme will be inhibited, or the consumption of the clock signaling enzyme will be promoted, or the activity of the clock signaling enzyme itself will be inhibited, resulting in the decrease of the concentration or activity of the clock signaling enzyme; when the concentration or activity of the anti-clock signaling enzyme is low, the generation of the clock signaling enzyme will be promoted, or the consumption of the clock signaling enzyme will be inhibited, or the activity of the clock signaling enzyme itself will be activated, resulting in the increase of the concentration or activity of the clock signaling enzyme.

On the other hand, the concentration or activity of the anti-clock signaling enzyme is affected by that of the clock signaling enzyme: when the concentration or activity of the clock signaling enzyme is high, the generation of the anti-clock signaling enzyme will be promoted, or the consumption of the anti-clock signaling enzyme will be inhibited, or the activity of the clock signaling enzyme itself will be activated, resulting in the increase of the concentration or activity of the anti-clock signaling enzyme; when the concentration or activity of the clock signaling enzyme is low, the generation of the anti-clock signaling enzyme will be inhibited, or the consumption of the anti-clock signaling enzyme will be promoted, or the activity of the clock signaling enzyme itself will be inhibited, resulting in the decrease of the concentration or activity of the clock signaling enzyme.

Because of the above interaction between the concentration or activity of the clock signaling enzyme and that of the anti-clock signaling enzyme, the clock and anti-clock signaling enzymes can realize the following negative-feedback oscillation regulation: When the concentration or activity of the clock signaling enzyme is high, the concentration or activity of the anti-clock signaling enzyme increases due to the influence of the clock signaling enzyme, and the concentration or activity of the anti-clock signaling enzyme increases to a high level; when the concentration or activity of the anti-clock signaling enzyme increases to a high level, the concentration or activity of the clock signaling enzyme decreases due to the influence of the anti-clock signaling enzyme, and the concentration or activity of the clock signaling enzyme decreases to a low level; when the concentration of the clock signaling enzyme decreases to a low level, the concentration or activity of the anti-clock signaling enzyme decreases due to the influence of the clock signaling enzyme, and the concentration or activity of the anti-clock signaling enzyme decreases to a low level; when the concentration or activity of the anti-clock signaling enzyme decreases to a low level, the concentration or activity of the clock signaling enzyme increases to a high level due to the influence of the anti-clock signaling enzyme; at this time, the clock oscillator completes an oscillation cycle, the clock signaling enzyme returning to the initial high-concentration state and being able to repeat the above cycle, thus realizing the oscillating output of the concentration or activity of the clock signaling enzyme with periodic fluctuations. The above negative-feedback regulation oscillation cycle can be succinctly shown in FIG. 2.

Optionally, the generation and consumption rate of the substance of the output signal is affected by the concentration or activity of the clock and anti-clock signaling enzymes, or the activity of the substance of the output signal is affected by the concentration or activity of the clock and anti-clock signaling enzymes. Therefore, the concentration or activity of the output signal can change with the concentration of the clock and anti-clock signaling enzymes, and the corresponding periodic oscillation signal can be outputted. Besides, changes in the concentration or activity of the output signal will not affect the concentration or activity of the clock and anti-clock signaling enzymes, thereby ensuring that the output feedforward propagation will not interfere with the oscillation cycle of the clock oscillator itself when the oscillation signal is outputted.

Optionally, the clock and anti-clock signaling enzymes can also be of the same substance, and the concentration or activity of the clock signaling enzyme is affected by that of its own: when the concentration or activity of the clock signaling enzyme is high, the generation of the clock signaling enzyme will be inhibited, or the consumption of the clock signaling enzyme will be promoted, or the activity of the clock signaling enzyme itself will be inhibited, resulting in the decrease of the concentration or activity of the clock signaling enzyme; when the concentration or activity of the clock signaling enzyme is low, the generation of the clock signaling enzyme will be promoted, or the consumption of the clock signaling enzyme will be inhibited, or the activity of the clock signaling enzyme itself will be activated, resulting in the increase of the concentration or activity of the clock signaling enzyme.

The present invention has the following advantages and effects with respect to the prior art:
(1) The clock oscillator for biochemical reaction computers disclosed by the present invention, with its output clock signal being a chemical signal of the concentration or activity of a substance or an enzyme, can be conveniently cascaded with the biochemical reaction logic gate and the biochemical reaction latch to form a timing logic, and can be used in the biochemical computer without the conversion between an electrical signal and the chemical signal.
(2) The clock oscillator for biochemical reaction computers disclosed by the present invention, due to the fast reaction speed of the enzymatic reaction on which it is based, has a higher clock frequency and a shorter clock cycle, and can realize the clock cycle of a millisecond to picosecond level.
(3) The clock oscillator for biochemical reaction computers disclosed by the present invention, with no need to rely on the DNA transcription and translation, can operate normally without relying on living cells, and can be used more flexibly.

### BRIEF DESCRIPTION OF THE DRAWINGS

FIG. 1 is a structural diagram of the clock oscillator for biochemical reaction computers disclosed in the present invention; and

FIG. 2 is a state transposition diagram of the oscillation cycle of the clock oscillator for biochemical reaction computers disclosed in the present invention.

### DETAILED DESCRIPTION OF THE EMBODIMENTS

In order to make the purpose, technical solution and advantages of the example of the present invention clearer, the technical solution in the example of the present invention will be described clearly and completely in the following in combination with the drawings in the example of the present invention. Obviously, the described example is only part, not all, of the examples of the present invention. Based on the example, all the other examples obtained by those skilled in the art without making creative efforts shall fall within the scope of protection of the present invention.

### Example

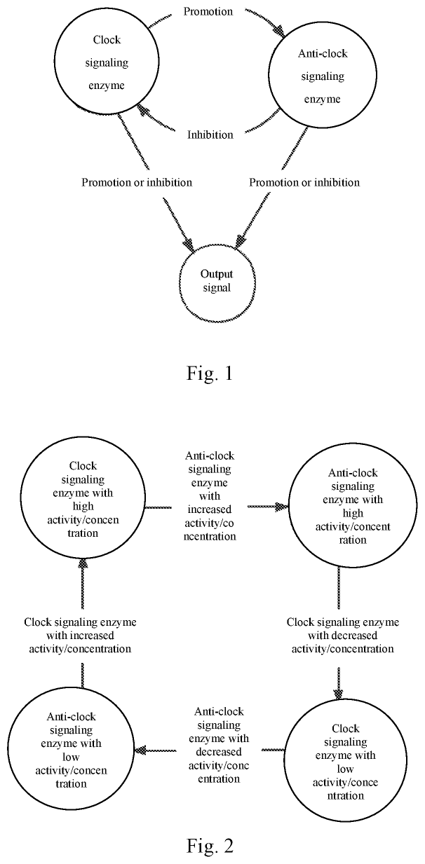

As shown in FIG. 1, this example provides a clock oscillator for biochemical reaction computers, which includes a clock signaling enzyme A and an anti-clock signaling enzyme B, and also involves the products of the clock signaling enzyme A and the anti-clock signaling enzyme B after inactivation, i.e. an inactivated clock signaling enzyme A′ and an inactivated anti-clock signaling enzyme B′. The clock signaling enzyme A and the inactivated clock signaling enzyme A′ can convert into each other. The anti-clock signaling enzyme B and the inactivated anti-clock signaling enzyme B′ can convert into each other. The conversion between these substances involves the following reactions:

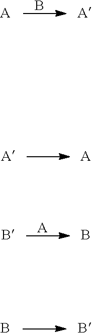

As shown in the above formulas, B can inhibit the activity of A by catalyzing the conversion of A into A′; without the participation of B, A′ can also spontaneously convert into A, thus regaining the activity; A can promote the activity of B′ by catalyzing the conversion of B′ into B; without the participation of A, B can also spontaneously convert into B′, thus losing the activity.

In the initial state, the concentration of A is high, that of A′ is low, that of B is low, and that of B′ is high. Because the concentration of A is high, B′ is converted into B under the catalysis of A, and the concentration of B increases; when the concentration of B increases to a high level, B starts to catalyze the conversion of A into A′, and the concentration of A decreases; when the concentration of A decreases to a low level, the catalytic conversion of B′ stops, while the spontaneous deactivation of B proceeds, making the concentration of B′ increase and the concentration of B decrease; when the concentration of B decreases to a low level, the catalytic deactivation of A stops, while the process of the spontaneous conversion of A′ into A to regain the activity proceeds, making the concentration of A′ decrease and the concentration of A increase to a high level; at this time, the concentration of A is high, that of A′ is low, that of B is low, and that of B′ is high, the concentrations of all the substances become the same as those in the initial state, and the concentration of the clock signaling enzyme changes from high to low and then to high, such that the clock oscillator completes an oscillation cycle and can continue the above cycle, so as to continuously output the periodically oscillating clock signal.

The realization of the signal oscillation of the clock oscillator usually depends on a negative-feedback regulation mechanism: When the value of the clock signal is large, the clock signal has a downward trend; when the value of the clock signal is small, the clock signal has an upward trend. The negative-feedback regulation mechanism of the existing biochemical reaction clock oscillators is often realized through the biological transcription system; that is, mRNA is generated through the DNA transcription, then mRNA is translated into a protein product, and then the protein product inhibits the DNA transcription, thus realizing the negative-feedback regulation. Due to the slow speed of the transcription and translation process, the existing biochemical reaction clock oscillators based on the transcription system have a long oscillation period. The clock oscillator provided by the present invention, based on the enzymatic reaction, controls the enzymatic reaction rate through the concentration or activity of the clock signaling enzyme, then controls the concentration or activity of the anti-clock signaling enzyme, and then controls the concentration or activity of the clock signaling enzyme through the concentration or activity of the anti-clock signaling enzyme, thereby realizing the negative-feedback regulation. Because the enzymatic reaction rate is high, the clock oscillator provided by the present invention can have a shorter oscillation period and a higher oscillation frequency, thus providing clock signals for high-performance biochemical reaction computers.

The above example is a preferred embodiment of the present invention, but the embodiments of the present invention are not limited thereto, and any other alterations, modifications, replacements, combinations, and simplifications made without departing from the spirit and principle of the present invention shall all be equivalent substitutions and included in the scope of protection of the present invention.

1. An enzymatic reaction-based clock oscillator for biochemical reaction computers, characterized in that: the clock oscillator comprises a clock signaling enzyme, an anti-clock signaling enzyme, and an output signal, wherein the concentration or activity of the clock signaling enzyme is affected by that of the anti-clock signaling enzyme, the concentration or activity of the anti-clock signaling enzyme is affected by that of the clock signaling enzyme, the generation and consumption rate of a substance of the output signal is affected by the concentration or activity of the clock and anti-clock signaling enzymes, or the activity of the substance of the output signal is affected by the concentration or activity of the clock and anti-clock signaling enzymes.

2. The clock oscillator for biochemical reaction computers according to claim 1, characterized in that: the concentration or activity of the clock signaling enzyme is affected by that of the anti-clock signaling enzyme: when the concentration or activity of the anti-clock signaling enzyme is high, the generation of the clock signaling enzyme will be inhibited, or the consumption of the clock signaling enzyme will be promoted, or the activity of the clock signaling enzyme itself will be inhibited, resulting in the decrease of the concentration or activity of the clock signaling enzyme; when the concentration or activity of the anti-clock signaling enzyme is low, the generation of the clock signaling enzyme will be promoted, or the consumption of the clock signaling enzyme will be inhibited, or the activity of the clock signaling enzyme itself will be activated, resulting in the increase of the concentration or activity of the clock signaling enzyme.

3. The clock oscillator for biochemical reaction computers according to claim 1, characterized in that: the concentration or activity of the anti-clock signaling enzyme is affected by that of the clock signaling enzyme: when the concentration or activity of the clock signaling enzyme is high, the generation of the anti-clock signaling enzyme will be promoted, or the consumption of the anti-clock signaling enzyme will be inhibited, or the activity of the clock signaling enzyme itself will be activated, resulting in the increase of the concentration or activity of the anti-clock signaling enzyme; when the concentration or activity of the clock signaling enzyme is low, the generation of the anti-clock signaling enzyme will be inhibited, or the consumption of the anti-clock signaling enzyme will be promoted, or the activity of the clock signaling enzyme itself will be inhibited, resulting in the decrease of the concentration or activity of the clock signaling enzyme.

4. The clock oscillator for biochemical reaction computers according to claim 1, characterized in that: the clock and anti-clock signaling enzymes are of the same substance or two different substances; when the clock and anti-clock signaling enzymes are of the same substance, the change of the concentration or activity of the clock signaling enzyme is affected by its current concentration or activity: when the concentration or activity of the clock signaling enzyme is high, the generation of the clock signaling enzyme will be promoted, or the consumption of the clock signaling enzyme will be inhibited, or the activity of the clock signaling enzyme will be inhibited, resulting in the decrease of the concentration or activity of the clock signaling enzyme; when the concentration or activity of the clock signaling enzyme is low, the generation of the clock signaling enzyme will be inhibited, or the consumption of the clock signaling enzyme will be promoted, or the activity of the clock signaling enzyme will be activated, resulting in the increase of the concentration or activity of the clock signaling enzyme.

5. The clock oscillator for biochemical reaction computers according to claim 4, characterized in that: when the same substance acts as the clock and anti-clock signaling enzymes, the concentration or activity of the clock signaling enzyme is affected by that of its own: when the concentration or activity of the clock signaling enzyme is high, the generation of the clock signaling enzyme will be inhibited, or the consumption of the clock signaling enzyme will be promoted, or the activity of the clock signaling enzyme itself will be inhibited, resulting in the decrease of the concentration or activity of the clock signaling enzyme; when the concentration or activity of the clock signaling enzyme is low, the generation of the clock signaling enzyme will be promoted, or the consumption of the clock signaling enzyme will be inhibited, or the activity of the clock signaling enzyme itself will be activated, resulting in the increase of the concentration or activity of the clock signaling enzyme.
